(19) United States
(12) Patent Application Publication
(10) Pub. No.: US 2020/0222362 A1
(43) Pub. Date: Jul. 16, 2020

(54) ORAL DISINTEGRATING FILMS FOR CANNABIS PRODUCTS

(71) Applicant: Tilray, Inc., Seattle, WA (US)

(72) Inventor: Harpreet KAUR, Nanaimo (CA)

(21) Appl. No.: 16/742,614

(22) Filed: Jan. 14, 2020

Related U.S. Application Data
(60) Provisional application No. 62/792,298, filed on Jan. 14, 2019.

(51) Int. Cl. A61K 31/352; A61K 9/70; A61K 9/00; A61K 31/05

(52) U.S. Cl. CPC A61K 31/352; A61K 31/05; A61K 9/0056; A61K 9/7007

(57) ABSTRACT

An oral disintegrating film including a cannabinoid or cannabinoid analogue, a first film forming polymer and a solubilizing agent are disclosed as well as methods of using same.

### CROSS-REFERENCE TO RELATED APPLICATION

This Application claims the benefit under 35 U.S.C. 119 (e) of, and priority to, U.S. Provisional Patent Application No. 62/792,298, filed Jan. 14, 2019, the entire contents of which are hereby explicitly incorporated by reference in their entirety.

### BACKGROUND

Oral delivery is a non-invasive and therefore a very convenient route of administration. Orally disintegrating dosage forms, like soluble films and (mini-)tablets, appear promising for use in the pediatric population. Of these, fast disintegrating films have superiority over fast disintegrating tablets as the latter are associated with risks of choking and friability. This drug delivery also has numerous advantages over conventional fast disintegrating tablets as they can be used for patients with dysphagia and schizophrenia without water due to their ability to disintegrate within a few seconds releasing medication in mouth.

Cannabinoids are natural extracts from the plant Cannabis sativa. The plant contains over hundred different compounds, but research has focused more on delta-9-tetrahydrocannabinol [THC] and cannabidiol [CBD]. THC is known to cause psychoactive effects or the ‘high’ felt from cannabis. THC has proven beneficial in patients suffering from Post-Traumatic Stress Disorder [PTSD], as an appetite stimulant for patients with HIV/AIDS, in reducing nausea and vomiting in patients on chemotherapy. On the other hand, CBD lacks nearly any psychoactive effect and has shown promise in treating epilepsy, including a severe form of epilepsy in children called Dravet's syndrome. CBD has also been used successfully by patients with genetic brain disorders, Crohn's disease and ulcerative colitis, and Parkinson's disease.

THC and CBD undergo hepatic-first pass metabolism hence delivery through the sublingual and buccal pathways is preferred to improve dosing and bioavailability, especially for patients requiring immediate relief.

Various approaches are employed for formulating orally disintegrating films (ODFs) and among which solvent casting, and spraying methods are frequently used. Generally, hydrophilic polymers along with other excipients are used for preparing ODFs which allow films to disintegrate quickly releasing incorporated API within seconds.

THC and CBD are lipophilic compounds which makes incorporation into hydrophilic polymers challenging. Additionally, they have a distinct bitter taste which makes them unpalatable to many patients.

There is a need for pharmaceutically acceptable oral disintegrating films that address the issues associated with incorporating lipophilic compounds that also have a bitter taste.

### SUMMARY OF THE INVENTION

The disclosure relates to oral disintegrating films that allow for administration of lipophilic compounds such as THC and CBD and also mask the bitter taste of the compounds. In accordance with one aspect, the present application discloses films based on self-emulsifying nano-emulsions (SEDDS). In accordance with certain aspects, SEDDS would serve as a carrier base for the drugs making incorporation into the film more feasible while also improving the stability of the drugs in the film. Furthermore, taste-masking would be easier with a SEDD system.

In another aspect, methods for administering lipophilic compounds such as CBD and THC are disclosed. The methods comprise administering the orally disintegrating film disclosed herein to a subject. In certain aspects, the present application relates to a method for the prevention and/or treatment of any one of the diseases or conditions mentioned herein comprising administering an orally disintegrating film containing a therapeutically effective amount of an active compound to a patient in need thereof.

In accordance with one aspect, the present application is directed to an oral disintegrating film including a cannabinoid or cannabinoid analogue, a first film forming polymer and a solubilizing agent. In some cases, the solubilizing agent has an HLB value between 10-20.

In some aspects, the solubilizing agent may be selected from the group consisting of PEG-32-stearate, lauroyl polyoxyl-32 glycerides NF, stearoyl polyoxyl-32 glycerides NF and mixtures thereof. In another aspect, the solubilizing agent is PEG-32-stearate.

In some aspects, the cannabinoid or cannabinoid analogue includes at least one of delta-9-tetrahydrocannabinol [THC] and cannabidiol [CBD].

In some aspects, the oral disintegrating film self-emulsifies in an aqueous medium to produce a plurality of particles having a mean particle size of about 1 to about 150 nm.

In some aspects, the oral disintegrating film further comprises a second film forming polymer.

In some aspects, the oral disintegrating film further comprises an excipient selected from the group consisting of a plasticizer, a taste masking agent, a flavor masking agent, a coloring agent, a flavorant, an effervescent agent, an organic acid, a pH modifying agent, and mixtures thereof.

In some aspects, the excipient includes an organic acid selected from the group consisting of sulfuric acid, nitric acid, hydrochloric acid, phosphoric acid, hydrobromic acid, acetic acid, p-toluenesulfonic acid, methanesulfonic acid, oxalic acid, maleic acid, fumaric acid, malic acid, citric acid, tartaric acid, succinic acid, ascorbic acid, adipic acid, lactic acid, and benzoic acid. In some cases, the excipient includes citric acid.

In some aspects, the cannabinoid or cannabinoid analogue is present in the film in an amount of from about 0.5 mg to 100 mg.

In some aspects, the solubilizing agent is present in the formulation in an amount of from about 5% to 80% based on weight.

In some aspects, the excipient includes a plasticizer selected from the group consisting of glycerin, medium chain glycerides (MCGs), long chain glycerides, propylene glycol esters and mixtures thereof. In certain cases, the plasticizer includes glycerin.

In some aspects, the oral disintegrating film has a thickness of from about 10 mm to 50 mm.

In some aspects, the oral disintegrating film has a weight of from about 30 mg to 200 mg.

In some aspects, the oral disintegrating film dissolves in less than 60 seconds in water at 23° C.

In accordance with another aspect, the present application is directed to an oral disintegrating film including a cannabinoid or cannabinoid analogue, a first film forming polymer, a second film forming polymer and a solubilizing agent, wherein the oral disintegrating film self-emulsifies in an aqueous medium.

In some aspects, each of the first film forming polymer and the second film forming polymer is selected from the group consisting of gelatin, pectin, hydroxypropylmethyl cellulose, methoxypolyethylene glycols, polyethylene glycols, hydroxyethyl cellulose, hydroxypropyl cellulose, carboxymethyl cellulose, polyvinylalcohol, polyacrylic acid, methyl methacrylate copolymer, polyvinyl caprolactam-polyvinyl acetate-polyethylene glycol graft co-polymer, carboxyvinyl polymer, polyethyleneglycol, alginic acid, sodium alginate, modified starch, casein, whey protein extract, soy protein extract, pea protein, rice, millet, buckwheat, tapioca, carboxymethyl/hydroxypropyl dual-modified tapioca, gelatinized tapioca starch, gelatinized potato starch, potato starch hydrolysates, legumes, zein, levan, elsinan, gluten, acacia gum, carageenan, Arabic gum, guar gum, locust bean gum, xanthan gum, gellan gum, agar and mixtures thereof.

In some aspects, the first film forming polymer may be selected from the group consisting of hydroxypropylmethyl cellulose, methoxypolyethylene glycols, Carbowax Sentry Polyethylene glycols, hydroxyethyl cellulose, hydroxypropyl cellulose, carboxymethyl cellulose, polyethylene glycols, hydroxyethyl cellulose, hydroxypropyl cellulose, carboxymethyl cellulose and mixtures thereof.

In some aspects, the second film forming polymer may be selected from the group consisting of gelatin, pectin, acacia gum, carageenan, Arabic gum, guar gum, locust bean gum, xanthan gum, gellan gum, agar and mixtures thereof.

In accordance with another aspect, the present application is directed to a method of reducing side effects associated with chemotherapy or radiation treatment, alleviating pain or suppressing appetite in a subject in need thereof comprising administering to the subject an oral disintegrating film as disclosed herein.

### DETAILED DESCRIPTION

As will be apparent to one of ordinary skill in the art from a reading of this disclosure, the disclosed subject matter can be embodied in forms other than those specifically disclosed herein. The particular embodiments described herein are, therefore, to be considered as illustrative and not restrictive. Those skilled in the art will recognize, or be able to ascertain, using no more than routine experimentation, numerous equivalents to the specific embodiments described herein.

### Definitions

The following are definitions of terms used in the present specification.

The term “film” includes films and sheets, in any shape, including rectangular, square or other shapes most appropriate for a specific application. The films described herein may be of any desired thickness and size suitable for the intended use. For example, a film of the present invention may be sized and shaped so that it may be easily placed into the oral cavity of the user to target a specific administration site for effective, localized delivery of active. In addition, some films may have a thickness of from about 10 to about 500 mm. In addition, the term “film” includes single layer compositions as well as multi-layer compositions, such as laminated films, coatings on films and the like.

The term “effective amount” or “therapeutically effective amount” refers to that amount of a compound or pharmaceutical composition described herein that is sufficient to effect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells. The specific dose will vary depending on, for example, the particular compounds chosen, the dosing regimen to be followed, whether it is administered in combination with other agents, timing of administration, the tissue to which it is administered, and the physical delivery system in which it is carried.

As used herein, the terms “treatment”, “treating”, “palliating” and “ameliorating” are used interchangeably. These terms refer to an approach for obtaining beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compositions can be administered to a patient at risk of developing a particular disease, or to a patient reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

In certain embodiments, the invention also relates to the use of a compound according to the invention, for the manufacture of a medicament for the treatment or prevention of any one of the diseases or conditions disclosed herein.

In view of the utility of the compounds according to the invention, there is provided a method of treating warm-blooded animals, including humans, suffering from any one of the diseases or conditions mentioned herein, and a method of preventing in warm-blooded animals, including humans, any one of the diseases or conditions mentioned herein.

Said methods comprise the administration, i.e. the systemic or topical administration, preferably oral administration, of a therapeutically effective amount of a compound according to the invention to warm-blooded animals, including humans.

Therefore, the invention also relates to a method for the prevention and/or treatment of any one of the diseases or conditions mentioned herein comprising administering a therapeutically effective amount of compound according to the invention to a patient in need thereof.

The formulations described herein can be used alone, in combination or in combination with other pharmaceutical agents such as other agents used in the treatment of various conditions. In such combinations, the compounds of the present invention may be utilized in combination with one or more other drugs in the treatment, prevention, control, amelioration, or reduction of risk of diseases or conditions for which compounds disclosed herein or the other drugs may have utility, where the combination of the drugs together are safer or more effective than either drug alone.

One skilled in the art will recognize that a therapeutically effective amount of the compounds of the present invention is the amount sufficient to bring about the intended effect and that this amount varies inter alia, depending on the type of disease, the concentration of the compound in the therapeutic formulation, and the condition of the patient. Generally, an amount of the compounds disclosed herein to be administered as a therapeutic agent for treating diseases and other conditions, such as the disorders described herein, may be determined on a case by case by an attending physician or other practitioner.

The amount of a compound according to the present invention, also referred to here as the active ingredient(s), which is required to achieve a therapeutic effect may vary on case-by-case basis, with the particular compound, the route of administration, the age and condition of the recipient, and the particular disorder or disease being treated. A method of treatment may also include administering the active ingredient on a regimen of between one and four intakes per day. In these methods of treatment, the compounds according to the invention are preferably formulated prior to admission. As described herein below, suitable pharmaceutical formulations are prepared by known procedures using well known and readily available ingredients.

The instant disclosure provides oral disintegrating films containing a cannabinoid or cannabinoid analogue. The films may also include components such as film forming polymers, plasticizers, solubilizing agents, organic acids, as well as other excipients. In certain aspects, the present application relates to oral disintegrating films utilizing a SEDD system. In accordance with one aspect, the present application provides formulations containing a cannabinoid or cannabinoid analogue in an oral disintegrating dosage form, such as a film, wherein the dosage form includes at least one surfactant, wherein the surfactant facilitates self-emulsification of the dosage form or film in an aqueous medium to produce a plurality of particles having a mean particle size of about 1 to about 150 nm, more particularly about 1 to about 50 nm, about 1 to 25 nm, and in some cases about 1 to 10 nm. In other cases, the particles have a mean particle size of about 10 to about 150 nm, more particularly about 25 to about 100 nm, and in some cases about 50 to 150 nm.

The term “analog” refers to a compound that is structurally related to naturally occurring cannabinoids, but whose chemical and biological properties may differ from naturally occurring cannabinoids. In the present context, analog or analogs refer to compounds that may not exhibit one or more unwanted side effects of a naturally occurring cannabinoid. Analog also refers to a compound that is derived from a naturally occurring cannabinoid by chemical, biological or a semi-synthetic transformation of the naturally occurring cannabinoid. Examples of these compounds include, but are not limited to, cannabinol, cannabidiol, Δ9-tetrahydrocannabinol, Δ8-tetrahydrocannabinol, 11-hydroxy-tetrahydrocannabinol, 11-hydroxy-Δ9-tetrahydrocannabinol, levonantradol, Δ-11-tetrahydrocannabinol, tetrahydrocannabivarin, dronabinol, amandamide, and nabilone. Moreover, any combination of two or more of the above mentioned cannabinoids can be present in the disclosed formulations. In accordance with certain aspects, the cannabinoid or cannabinoid analogue comprises at least one of delta-9-tetrahydrocannabinol [THC] and cannabidiol [CBD].

The cannabinoid and/or cannabinoid analogue may be present in the formulation or other composition in an amount of from about 0.25 to 200, more particularly from about 0.5 to 100, still more particularly from about 1 to 50, and in certain cases from about 5 to 40 mg active per film. As a percentage based on the total weight of the film, the cannabinoid and/or cannabinoid analogue may be present in an amount of from about 0.25 to 50%, more particularly from about 0.5 to 37.5%, still more particularly from about 1 to 25% by weight. In accordance with certain embodiments, the active includes CBD and/or THC. In some cases, the CBD and THC are both present and in certain cases present in equal amounts.

In accordance with some embodiments, the ODF of the present invention comprises one or more film-forming polymers. The film-forming polymers may dissolve easily in solvents such as water such that the film dissolves rapidly in the buccal cavity. The film forming polymer(s) may be included in an amount of 10%-75% by weight, more particularly from about 15%-50%, and still more particularly from about 20%-40% based on the total weight of the orally fast dissolving film formulation.

Exemplary film forming polymers include but are not limited to gelatin, pectin, low viscosity pectin, hydroxypropylmethyl cellulose (HPMC), methoxypolyethylene glycols, low viscosity hydroxylpropylmethyl cellulose, Carbowax Sentry polyethylene glycols, hydroxyethyl cellulose, hydroxypropyl cellulose, carboxymethyl cellulose, polyvinylalcohol, polyacrylic acid, methyl methacrylate copolymer, polyvinyl caprolactam-polyvinyl acetate-polyethylene glycol graft co-polymer, carboxyvinyl polymer, EUDRAGIT® E, EUDRAGIT® L and EUDRAGIT® FS polymers, polyethyleneglycol, alginic acid, low viscosity alginic acid, sodium alginate, modified starch, casein, whey protein extract, soy protein extract, pea protein, rice, millet, buckwheat, tapioca, Carboxymethyl/Hydroxypropyl Dual-Modified Tapioca, gelatinized tapioca starch, gelatinized potato starch, potato starch hydrolysates, legumes, zein, levan, elsinan, gluten, acacia gum, carageenan, Arabic gum, guar gum, locust bean gum, xanthan gum, gellan gum, agar and combinations thereof. Examples of HPMC are HPMC with viscosity from about 3 cps to about 100,000 cps and, more specifically, HPMC 2600-5600 cps. In accordance with certain embodiments, a combination of film forming polymers can be used to provide the desired properties for the film.

Plasticizers that are useful in the present formulation include those that can solubilize or assist in solubilizing the active(s) or provide a stabilizing effect. Examples of suitable plasticizers include, but are not limited to, medium-chain glycerides, a long-chain glycerides, propylene glycol fatty acid esters and mixtures thereof.

Medium-chain glycerides (MCGs) contain 6-12 carbon fatty acid esters of glycerol and may be a mono-, di- or triglyceride. Particularly useful MCGs include medium chain triglycerides. Other useful MCGs include caprylic and capric mono- and diglycerides, and blends thereof, including glyceryl monocaprylate, glyceryl dicaprylate, glyceryl monocaprate and glyceryl dicaprate. Other MCGs include caprylic/capric triglycerides, glycerol esters of lauric acid, such as glyceryl monolaurate, glyceryl dilaurate and glycerol trilaurate, and polyglycerol esters of caprylic acid.

Long-chain glycerides (LCGs) contain 14-22 carbon fatty acid esters of glycerol. The LCG may be a mono-, di- or triglyceride. Examples of LCGs include glyceryl behenate, glyceryl monolinoleate, glycerol monooleate, glycerol monostearate, glycerol monopalmitate, glyceryl dilinoleate, glycerol diooleate, glycerol distearate, glycerol dipalmitate, glyceryl trilinoleate, glyceryl triolein, glyceryl tristearate, glyceryl tripalmitate. Other examples of LCGs include simple oils including, but not limited to the following: jojoba oil, almond oil, canola oil, castor oil, cod liver oil, corn oil, cottonseed oil, evening primrose oil, fish oil, grape seed oil, olive oil, palm kernel oil, palm oil, peanut oil, rapeseed oil, safflower oil, sesame oil, soybean oil, sunflower oil, hydrogenated castor oil, hydrogenated coconut oil, hydrogenated cottonseed oil, hydrogenated palm oil, hydrogenated soybean oil, partially hydrogenated soybean oil and hydrogenated vegetable oil.

Examples of propylene glycol fatty acid esters that may be used in the formulation as the lipid component include propylene glycol monocaprylate, propylene glycol dicaprate, propylene glycol monolaurate, propylene glycol dilaurate, and propylene glycol heptanoate. These propylene glycol fatty acid esters may also be used in the formulations as surfactants.

When targeting the development of a plasticizer solution, particularly useful plasticizer components in the present invention include those that can dissolve the compound at concentrations greater than 100 mg active per gram of excipient (“mg/g” hereinafter), and more particularly, above 150 mg/g, and still more particularly, above 500 mg/g, which may be determined via solubility assays. For cannabinoids, particularly useful plasticizer components include glycerin, medium-chain triglycerides, phospholipids, phospholipid derivatives, vitamin E derivatives, glyceryl dibehanate, behenoyl polyoxyl-8-glycerides, Geloil SC (soybean oil glyceryl palmitostearate), glyceryl monostearate PEG-75 stearate and combinations thereof.

The surfactant or solubilizing agent has a capacity to emulsify the plasticizer component of the formulation and has a hydrophilic-lipophilic balance (“HLB”) of at least 1, more particularly at least 18. In some cases, the HLB for the surfactants in the formulation is between 10 and 16. The hydrophilic-lipophilic balance of a surfactant is a measure of the degree to which it is hydrophilic or lipophilic, determined by calculating values for the different regions of the molecule. An HLB value of 0 corresponds to a completely lipophilic/hydrophobic molecule, and a value of 20 corresponds to a completely hydrophilic/lipophobic molecule. HLB values for various surfactants are well known in the art.

In accordance with certain embodiments, the surfactant or solubilizing agent may be selected from propylene glycol mono- or diesters of 8-22 carbon fatty acids, sorbitan fatty acid esters including sorbitan monolaurate; polyoxyethylene sorbitan fatty acid esters such as polysorbate 20, polysorbate 40, polysorbate 60, and polysorbate 80, polysorbate 85; polyoxyethylated mono- and di-fatty acid esters such as esters of castor oil (Kolliphor® EL), hydrogenated castor oil (Kolliphor® RH40), hydroxystearic acid (Kolliphor® HS-15); glycerol macrogolglycerides such as Labrasol®, Gelucire® 44/14, Gelucire® 50/13, Labrafil®; DL-α-tocopheryl polyethylene glycol succinate; polyoxyethylene-polyoxypropylene copolymers such as poloxamer 124, poloxamer 188, poloxamer 407; polyglycerol esters of fatty acids such as polyglycerol-6-caprylate, polyglycerol-3-oleate; and ethoxylated fatty alcohols such as the Brij® surfactants.

Particularly useful surfactants or solubilizing agents in the present formulation are those with a capacity to emulsify the plasticizer component of the formulation, namely those surfactants, particularly non-ionic surfactants, with a HLB greater than 8 for example Span 20 and polysorbate 85 (Tween 85), or, more particularly, a HLB greater than 12, for example, Kolliphor® RH40 (also known as Macrogolglycerol hydroxystearate, PEG-40 castor oil, Polyoxyl 40 hydrogenated castor oil), Kolliphor® EL (also known as Macrogolglycerol ricinoleate, PEG-35 castor oil, Polyoxyl 35 hydrogenated castor oil, Polyoxyl-35 castor oil), Kolliphor® HS 15 (also known as Macrogol (15)-hydroxystearate, Polyethylene glycol (15)-hydroxystearate, Polyoxyethylated 12-hydroxystearic acid, Solutol® HS 15), polysorbate 20 (also known as polyethylene glycol sorbitan monolaurate, polyoxyethylene sorbitan monolaurate, TWEEN® 20), polysorbate 60 (also known as Polyethylene glycol sorbitan monostearate, Polyoxyethylene sorbitan monostearate, TWEEN® 60), polysorbate 80 (also known as Polyoxyethylenesorbitan monooleate, TWEEN® 80), Gelucire® 44/14 (Lauroyl Polyoxyl-32 glycerides), Gelucire® 48/16 (polyethylene glycol monostearate, PEG-32 stearate, polyoxylethylene stearates), Labrasol® (Caprylocaproyl Polyoxyl-8 glycerides), Gelucire® 50/13 (Stearoyl polyoxyl-32 glycerides, Stearoyl polyoxylglycerides NF, Stearoyl macrogolglycerides EP) or Vitamin E TPGS DL-α-tocopheryl polyethylene glycol succinate. In one embodiment, the surfactant may be one or more non-ionic surfactants. In addition to those non-ionic surfactants previously disclosed, additional examples of non-ionic surfactants that may be used in certain embodiments include, but are not limited to, polyoxyethylated mono- and di-fatty acid esters of castor oil or hydrogenated castor oil, and polyethylene glycol ester of caprylic/capric glycerides, and sorbitan monolaurate, and blends thereof. It is also possible to utilize a single surfactant or a combination of lipophilic and hydrophilic surfactants in a cannabinoid lipid formulation as disclosed herein. Examples of particularly useful surfactants include polyethoxylated castor oil, such as polyoxyl 35 castor oil, poloxamers, hydrogenated castor oil ethoxylates, polyoxylethylene stearates, polyoxyl glycerides, glycol monolaureate, polyglyceryl dioleate and combinations thereof. Kolliphor® EL and Gelucire® 48/16 are particularly useful. Kolliphor® EL is a nonionic solubilizer and emulsifier made by reacting castor oil with ethylene oxide in a molar ratio of 1:35. Kolliphor® EL includes glycerol polyethylene glycol ricinoleate with fatty acid esters of polyethylene glycol and free polyethylene glycols and ethoxylated glycerol. Gelucire® 48/16 contains PEG-32 (MW 1500) esters of palmitic (C16) and stearic (C18) acids. In accordance with certain embodiments, the formulation is free of cationic and/or anionic surfactants.

The amount of plasticizer and surfactant or solubilizing agent in the ODF may be chosen so as to enable relatively high compound loadings of cannabinoid with acceptable formulation dispersibility. In general, the formulation or composition contains between 0.1-10% w/w, 0.5-7.5% w/w, typically 0.75-5% w/w plasticizer component and 0.01-80%, more particularly 0.1 to 60% w/w, typically 0.5 to 40%, 1 to 25% w/w, typically 5 to 15%, w/w non-ionic surfactant or solubilizing agent. In general, the ratio of plasticizer component to surfactant is at least 0.1:1. The ratio may be at least 0.1:1, may be at least 1:1, may be at least 1.5:1, may be at least 2:1, or may be at least 3:1.

The ODF may contain other optional excipients or other components. These optional excipients or other components may be included to provide various benefits such as improving emulsification of the lipid component in the formulation and overall drug solubility. Examples of optional excipients or other components may include phospholipids, free fatty acids, fatty acid alcohols or synthetic fatty acid derivatives including isopropyl myristate and isopropyl palmitate. Isopropyl myristate and isopropyl palmitate may also be added to the lipid formulation as a cosolvent, for the purpose of improving drug solubility in the formulation. Other example cosolvents may include propylene glycol, polyethylene glycol, triacetin, glycerol, ethanol and diethylene glycol monoethyl ether, or other pharmaceutically acceptable cosolvents.

The ODF may also include other excipients including, but not limited to, a taste masking agent, a flavor masking agent, a coloring agent, a flavorant, an effervescent agent, an organic acid, a pH modifying agent, and mixtures thereof.

Since the CBD and THC have a very bitter taste, the film may also contain one or more taste masking agents or bitter blockers. The amount of the taste masking agents may range from about 0.001% to about 0.5% by weight of the film and may be selected from the group of kleptose, cyclodextrin, cyclodextrin derivatives, ginger, anise, cinnamon, peppermint, licorice, fruit juice, sweeteners, sucrose, glucose, fructose, mannitol, saccharin, aspartame, sucralose, stevia plant derivatives, honey, or any combination thereof.

The ODF may also contain an organic acid. The organic acid lowers the pH of the orally fast dissolving film formulation to increase the solubility of the active, contributing to an improvement in the dissolution rate of the film. Other roles of the organic acid are to promote the secretion of saliva in the mouth and to impart a sour taste to the orally fast dissolving film formulation, allowing a taker to be less sensitive to bitterness peculiar to the active(s). Examples of organic acids include, but are not necessarily limited to, sulfuric acid, nitric acid, hydrochloric acid, phosphoric acid, hydrobromic acid, acetic acid, p-toluenesulfonic acid, methanesulfonic acid, oxalic acid, maleic acid, fumaric acid, malic acid, citric acid, tartaric acid, succinic acid, ascorbic acid, adipic acid, lactic acid, and benzoic acid.

In accordance with certain embodiments, the organic acid may be an acid derived from food and may be, for example, selected from citric acid, acetic acid, maleic acid, lactic acid, tartaric acid, ascorbic acid, adipic acid, succinic acid, fumaric acid, and mixtures thereof. In accordance with certain embodiments, the organic acid is citric acid or tartaric acid. The food-derived organic acid is effective in promoting the secretion of saliva in the mouth of a patient, enabling the patient to take the orally fast dissolving film without water, and serves to prevent the intra-oral pH from being excessively lowered.

The organic acid, such as citric acid, may be included in an amount of 0.001%-10% by weight, more particularly from about 0.01%-7.5%, and still more particularly from about 0.1%-5% based on the total weight of the orally fast dissolving film formulation.

The orally fast dissolving film formulation of the present invention may further include a sweetening agent. The sweetening agent can also mask a bitter taste. Examples of useful sweetening agents include, but are not limited to, sucralose, acesulfame potassium, L-menthol, xylitol, aspartame, saccharin salts, neotame, cyclamate salts, thaumatin, Luo han guo extract, licorice extract, sugar, glucose, maltose, oligosaccharides, dextrin, invert sugar, fructose, lactose, galactose, starch syrup, sorbitol, maltitol, erythritol, hydrogenated starch syrup, mannitol, and trehalose. These sweetening agents may be used alone or as a mixture thereof. The sweetening agent, when present, may be included in an amount of 0.5 to 5.0% by weight, based on the total weight of the ODF.

The ODF disclosed herein allows for administration in the absence of water or fluid intake. The ODF of the present invention is fast acting due to characteristics such as fast disintegration, dissolution and permeation rates. Specifically, the fast acting ODF of the present invention disintegrates in the saliva in less than about 60 seconds, more particularly in less than 45 seconds and in some cases less than 30 seconds.

In accordance with certain embodiments, the films may include the following ingredients within one or more of the exemplary ranges as set forth in Tables 1 and 2. These ranges are example ranges only and should not be considered limiting. Furthermore, a particular ingredient falling within one range can be combined with another ingredient falling within a different range. Ranges for a particular component can also be combined. For example, a lower limit of range may be combined with an upper range for another range.

TABLE 1
(% by weight)

| Ingredients | Broad | Intermediate | Narrow |
|---|---|---|---|
| Active | 0.25%-50% | 0.5%-37.5% | 1%-25% |
| Film Forming | 10%-75% | 15%-50% | 20%-37.5% |
| Polymer | | | |
| Plasticizer | 0.1%-10% | 0.5%-7.5% | 0.75%-5% |
| Solubilizing | 0.01%-80% | 0.1%-60% | 0.5%-40% |
| Agent | | | |
| Organic Acid | 0.001%-10% | 0.01%-7.5% | 0.1%-5% |
| Other | As needed | As needed | As needed |

TABLE 2

| Ingredients | Broad | Intermediate | Narrow |
|---|---|---|---|
| Active (e.g., THC) | 0.5-100 | 1-75 | 1.5-50 |
| | mg/film | mg/film | mg/film |
| Active (e.g., CBD) | 0.5-100 | 1-75 | 1.5-50 |
| | mg/film | mg/film | mg/film |
| First Film Forming | 1 mg-175 | 10 mg-125 | 15 mg-100 |
| Polymer (e.g., HPMC) | mg/film | mg/film | mg/film |
| Second Film Forming | 1 mg-100 | 10 mg-90 | 15 mg-75 |
| Polymer (e.g., Pectin, | mg/film | mg/film | mg/film |
| Gelatin) | | | |
| Solubilizing Agent (e.g., | 0.1 mg-100 | 1 mg-90 | 10-75 |
| Gelucire 48/16) | mg/film | mg/film | mg/film |
| Organic Acid (e.g., | 0.0001-20 | 0.001-15 | 0.01 mg-12.5 |
| Citric Acid) | mg/film | mg/film | mg/film |
| Other (e.g., Menthol) | 0.0001-20 | 0.001-15 | 0.01 mg-12.5 |
| | mg/film | mg/film | mg/film |

Methods of producing the ODF are not particularly limited. In accordance with one aspect, the ODF can be produced in accordance with the following process. Film forming polymer is dissolved in a suitable volatile solvent. The active is entrapped in solubilizer and plasticizers until it forms a uniform dispersion. This active dispersion is then gradually suspended in the polymer matrix under controlled manufacturing conditions. The polymer matrix now infused with active is dried and yields a uniform film. The film can be cut into various shapes and dimensions as required.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification and guidance which can be adapted to the practice of this invention in its various embodiments and equivalents thereof.

### Examples

TABLE 3
Multiple formulation compositions were tested:

| Example 1 | | Example 2 | |
|---|---|---|---|
| Ingredients | mg/Film | Ingredients | mg/Film |
| Gelatin | 35.5 | Kollicoat IR | 38.9 |
| Glycerin | 11.15 | PEG 300 | 8 |
| Kolliphor EL | 2.5 | Kolliphor P 407 | 2.5 |
| Citric acid | 0.5 | Citric acid | 0.5 |
| Sucralose | 0.1 | Sucralose | 0.1 |
| Peppermint oil | 0.25 | | |
| Total Qty. | 50 | Total Qty. | 50 |
| Parameters | Example 1 | Parameters | Example 2 |
| | Observations | | Observations |
| Vacuum Oven | 24 hr at 45° C. in | Vacuum Oven | 1 hr at 60° C. in |
| drying | | drying | |
| Air drying | 18 hr at RT | Air drying | 18 hr at RT |
| Visual | Transparent and | Visual | Transparent and |
| appearance | flexible | appearance | flexible |
| of the film | | of the film | |
| Dispersion test | 121.7 sec | Dispersion test | 27.3 sec |
| Average | 0.09 mm | Average | 0.08 mm |
| Thickness | | Thickness of | |
| of the film | | the film | |
| Average | 44.1 mm | Average | 50.1 mm |
| Weight of | | Weight of | |
| the film | | the film | |

Note:
Low drying temp is used to prevent melting of Gelatin

The dispersion test is determined by measuring the time taken for the film to dissolve completely in 25 ml water at room temperature (23° C.).

A few other combinations of polymer and surfactant were also evaluated as set forth in Table 4.

TABLE 4

| Example 3 | | Example 4 | | Example 5 | | Example 6 | |
|---|---|---|---|---|---|---|---|
| Ingredients | mg/Film | Ingredients | mg/Film | Ingredients | mg/Film | Ingredients | mg/Film |
| Kollicoat | 37.5 | HPMC E 5 | 37.5 | Kollicoat | 42.5 | Kollicoat | 32.5 |
| IR | | | | IR | | IR | |
| Gelucire | 5 | Gelucire | 5 | Gelucire | 0 | Gelucire | 10 |
| 48/16 | | 48/16 | | 48/16 | | 48/16 | |
| Kolliphor P | 2.5 | Kolliphor P | 2.5 | Kolliphor P | 2.5 | Kolliphor P | 2.5 |
| 407 | | 407 | | 407 | | 407 | |
| Citric acid | 2 | Citric acid | 2 | Citric acid | 2 | Citric acid | 2 |
| Sucralose | 3 | Sucralose | 3 | Sucralose | 3 | Sucralose | 3 |
| Total Qty. | 50 | Total Qty. | 50 | Total Qty. | 50 | Total Qty | 50 |

These examples were all formed using the solvent casting method. The films were too fragile-cracking upon slight touch and not flexible at all. Hence, different excipients were evaluated.

Kollicoat IR, HPMC E5, Methyl cellulose and Pectin were evaluated as film forming agents. Lactose was used to enable burst release of the film. Kolliphor ER, Gelucire 48/16 are key components of the SEDDS. Citric acid was incorporated stimulate the salivary production in oral cavity.

TABLE 5

| | Example 7 | Example 8 | Example 9 |
|---|---|---|---|
| Ingredients | [mg/film] | [mg/film] | [mg/film] |
| Kollicoat IR | 18 | — | — |
| HPMC E5 | — | 18 | — |
| Methyl | — | — | 18 |
| cellulose | | | |
| Pectin | 5 | 5 | 5 |
| Lactose | 14 | 14 | 14 |
| Poloxamer | 3 | 3 | 3 |
| 188 | | | |
| Kolliphor EL | 3 | 3 | 3 |
| Gelucire | 5 | 5 | 5 |
| 48/16 | | | |
| Citric acid | 2 | 2 | 2 |

| | | |
|---|---|---|
| Observation | Forms a good film | Films formed are |
| #1 | but it is brittle | exceptionally good, need |
| | probably due to | optimization once drug |
| | lack of viscosity | is incorporated as drug is |
| | imparting agent. | highly viscous and lipophilic |

| | |
|---|---|
| Observation | Dissolves in less than 30 seconds when placed |
| #2 | in 25 ml of water |
| Conclusion | Kollicoat is a good film forming agent but makes the films |
| | brittle if used alone potentially due to lack of a viscosity |
| | imparting plasticizer. |
| | Hence combination of polymers to be evaluated - |
| | HPMC E5, Pectin and Methyl Cellulose. |

The following examples are directed to compositions exhibiting improved taste-masking of the film. Kollicoat SmartSeal 30D is a quick dissolving film and manufactured by BASF for taste-masking and protection from moisture. It is a reverse-enteric polymer that solubilizes at pH 5.5 and is insoluble in pH above 5.5 units. pH of the saliva is around 6.5 pH units; hence the goal was broken down into sub-goals—

1. Check if Kollicoat SmartSeal 30D is a good film former and can be used with the existing polymers and plasticizers

2. If yes, incorporate with drug and charge on stability.

A series of trials were conducted with different compositions of Kollicoat SmartSeal 30D, Pectin, and Beta-cyclodextrin. Beta-cyclodextrin is a complexing agent and was evaluated as a potential carrier.

Different permutation combinations of KSS and Beta-cyclodextrin were evaluated. The films that formed were too crisp/fragile and not uniform.

TABLE 6

| | Exam- | Exam- | Exam- | Exam- |
|---|---|---|---|---|
| | ple 10 | ple 11 | ple 12 | ple 13 |
| Ingredients | [mg/film] | [mg/film] | [mg/film] | [mg/film] |
| Kollicoat ® | 37.33 | 18.67 | 31.12 | 24.89 |
| Smartseal 30 D | | | | |
| beta cyclodextrin | 10.67 | 32 | 10.67 | 32 |
| pectin | 13.33 | 6.67 | 11.11 | 8.89 |
| kolliphor EL | 13.33 | 6.67 | 11.11 | 8.88 |
| PEG 3350 | 5.33 | 16 | 16 | 5.33 |
| Total Qty. | 80 | 80 | 80 | 80 |

Hence, beta-cyclodextrin was removed from the next set of trials.

TABLE 7
[mg/film]
Example 14

| | |
|---|---|
| Kollicoat | 50 |
| Smartseal 30D | |
| Pectin | 10 |
| Gelucire 48/16 | 5 |
| Kolliphor EL | 5 |
| Citric Acid | 3 |
| Sucralose | 3 |
| Menthol | 2 |
| Total Qty. | 78 |

Example 15

| | |
|---|---|
| Kollicoat | 40 |
| Smartseal 30D | |
| Pectin | 10 |
| Gelucire 48/16 | 5 |
| KEL | 5 |
| Citric Acid | 3 |
| Sucralose | 3 |
| Menthol | 2 |
| PEG 4000 | 10 |
| Total Qty. | 78 |

Example 16

| | |
|---|---|
| Kollicoat | 40 |
| Smartseal 30D | |
| Pectin | 5 |
| Gelucire 48/16 | 5 |
| KEL | 5 |
| Citric Acid | 3 |
| Sucralose | 3 |
| Menthol | 2 |
| PEG 4000 | 15 |
| Total Qty. | 78 |

Example 17

| | |
|---|---|
| Kollicoat | 30 |
| Smartseal 30D | |
| Pectin | 10 |
| Gelucire 48/16 | 5 |
| KEL | 5 |
| Citric Acid | 3 |
| Sucralose | 3 |
| Menthol | 2 |
| PEG 4000 | 20 |
| Total Qty. | 78 |

It was identified that Kollicoat SmartSeal 30D, precipitated upon drying.

The compositions disclosed below are directed to food-grade films. Pectin is a commonly used film forming agent, and hence was evaluated in the formation of the orally disintegration film.

TABLE 8

| | Example 18 | | | Example 9 | | | Example 20 | |
|---|---|---|---|---|---|---|---|---|
| | mg/film | % | | mg/film | % | | mg/film | % |
| Pectin | 10 | 0.13 | Pectin | 5 | 0.06 | Pectin | 10 | 0.13 |
| Gelucire | 5 | 0.06 | Gelucire | 5 | 0.06 | Gelucire | 5 | 0.06 |
| 48/16 | | | 48/16 | | | 48/16 | | |
| KEL | 5 | 0.06 | KEL | 5 | 0.06 | KEL | 5 | 0.06 |
| Citric | 3 | 0.04 | Citric | 3 | 0.04 | Citric | 3 | 0.04 |
| Acid | | | Acid | | | Acid | | |
| Sucralose | 3 | 0.04 | Sucralose | 3 | 0.04 | Sucralose | 3 | 0.04 |
| Menthol | 2 | 0.03 | Menthol | 2 | 0.03 | Menthol | 2 | 0.03 |
| PEG | 10 | 0.13 | PEG | 15 | 0.19 | PEG | 20 | 0.26 |
| 4000 | | | 4000 | | | 4000 | | |
| Total | 38 | | Total | 38 | | Total | 48 | |
| Quantity | | | Quantity | | | Quantity | | |

The following examples are directed to films with drug encapsulation.

TABLE 9

| | Example 21 | |
|---|---|---|
| Ingredients | mg/Film | % w/w |
| THC | 10.0 | 12.5 |
| CBD | 10.0 | 12.5 |
| HPMC E 5 | 20.0 | 25.0 |
| Pectin | 5.0 | 6.25 |
| Lactose | 14.0 | 17.5 |
| Poloxamer 188 | 5.0 | 6.25 |
| Kolliphor EL | 5.0 | 6.25 |
| Gelucire 48/16 | 5.0 | 6.25 |
| Citric acid | 3.0 | 3.75 |
| Sucralose | 3.0 | 3.75 |
| Total | 80.0 | 100 |

Observation: Example 21: Films produced semi-transparent and slightly brownish in color. Films are flexible but crack after folding twice or thrice from the same place. Need to improve flexibility of the films. Films contain air bubbles due to improper processing.

TABLE 10

| | Example 22 | |
|---|---|---|
| Ingredients | mg/Film | % w/w |
| THC | 10.0 | 12.5 |
| CBD | 10.0 | 12.5 |
| Methyl cellulose | 20.0 | 25.0 |
| Pectin | 5.0 | 6.25 |
| Lactose | 14.0 | 17.5 |
| Poloxamer 188 | 5.0 | 6.25 |
| Kolliphor EL | 5.0 | 6.25 |
| Gelucire 48/16 | 5.0 | 6.25 |
| Citric acid | 3.0 | 3.75 |
| Sucralose | 3.0 | 3.75 |
| Total | 80.0 | 100 |

### Observation:

Films are semi-transparent and slightly brownish in color with air bubbles. Films are more flexible as compared to Example 21. These films dissolve in 1 minute in water.

Table 11 presents a additional formulations, which were tested and provided high quality films.

TABLE 11

| | Example 23 | | | Example 24 | | | Example 25 | |
|---|---|---|---|---|---|---|---|---|
| | mg/film | % | | mg/film | % | | mg/film | % |
| HPMC | 0.5 | 1 | HPMC | 0.5 | 1 | HPMC | 0.5 | 1.96 |
| Gelatin | 24.5 | 49 | Pectin | 24.5 | 49 | Pectin | NA | NA |
| Glycerin | 2 | 4 | Glycerin | 2 | 4 | Glycerin | 2 | 7.843 |
| Gelucire | 20 | 40 | Gelucire | 20 | 40 | Gelucire | 20 | 78.483 |
| 48/16 | | | 48/16 | | | 48/16 | | |
| Citric Acid | 1 | 2 | Citric Acid | 1 | 2 | Citric Acid | 1 | 3.921 |
| Menthol | 1 | 4 | Menthol | 1 | 4 | Menthol | 2 | 7.843 |
| Total | 50 | | Total | 50 | | Total | 25.5 | |
| Quantity | | | Quantity | | | Quantity | | |

It was surprising to note that Example 25 yielded the best quality of film. It is hypothesized that the critical characteristics of Gelucire 48/16 help with the formation of the film with the minimal quantity of HPMC. Citric acid is incorporated to help with salivation and wetting of the film—to dissolve the film quickly.

TABLE 12

| | Example 26 | Example 27 | Example 28 | Example 29 | Example 30 | Example 31 | Example 32 |
|---|---|---|---|---|---|---|---|
| Ingredients | mg/film | mg/film | mg/film | mg/film | mg/film | mg/film | mg/film |
| CBD | 2.5 | 2.5 | 2.5 | 2.5 | 2.5 | 2.5 | 2.5 |
| THC | 2.5 | 2.5 | 2.5 | 2.5 | 2.5 | 2.5 | 2.5 |
| pectin | 24.5 | 18.38 | 12.25 | 36.75 | 36.75 | 36.75 | 36.75 |
| Citric Acid | 1 | 0.75 | 0.5 | 1.5 | 1.5 | 1.5 | 1.5 |
| Menthol | 2 | 1.5 | 1 | 3 | 3 | 3 | 3 |
| Gelucire 48/16 | 20 | 15 | 10 | 30 | 30 | 30 | 30 |
| HPMC 2600-5600 cp | 0.5 | 0.38 | 0.25 | 1.5 | 3 | 0.75 | 0.75 |
| glycerin | 2 | 1.5 | 1 | 3 | 3 | 7.5 | 12 |
| Total | 55 | 42.5 | 30 | 80.75 | 82.25 | 84.5 | 89 |

Example 32 was developed to address the concerns of water solubility, content uniformity and potency using natural ingredients. A 1:1 ratio of Pectin with Gelucire, with a higher glycerin content improved dissolution of the film in the water.

1. An oral disintegrating film comprising:
a cannabinoid or cannabinoid analogue;
a first film forming polymer; and
a solubilizing agent, wherein said solubilizing agent has an HLB value between 10-20.

2. The oral disintegrating film of claim 1, wherein the solubilizing agent is selected from the group consisting of PEG-32-stearate, lauroyl polyoxyl-32 glycerides NF, stearoyl polyoxyl-32 glycerides NF and mixtures thereof.

3. The oral disintegrating film of claim 1, wherein the solubilizing agent comprises PEG-32-stearate.

4. The oral disintegrating film of claim 1, wherein the cannabinoid or cannabinoid analogue comprises at least one of delta-9-tetrahydrocannabinol [THC] and cannabidiol [CBD].

5. The oral disintegrating film of claim 1, wherein the oral disintegrating film self-emulsifies in an aqueous medium to produce a plurality of particles having a mean particle size of about 1 to about 150 nm.

6. The oral disintegrating film of claim 1, wherein the oral disintegrating film further comprises a second film forming polymer.

7. The oral disintegrating film of claim 1, wherein the oral disintegrating film further comprises an excipient selected from the group consisting of a plasticizer, a taste masking agent, a flavor masking agent, a coloring agent, a flavorant, an effervescent agent, an organic acid, a pH modifying agent, and mixtures thereof.

8. The oral disintegrating film of claim 7, wherein the excipient comprises an organic acid selected from the group consisting of sulfuric acid, nitric acid, hydrochloric acid, phosphoric acid, hydrobromic acid, acetic acid, p-toluenesulfonic acid, methanesulfonic acid, oxalic acid, maleic acid, fumaric acid, malic acid, citric acid, tartaric acid, succinic acid, ascorbic acid, adipic acid, lactic acid, and benzoic acid.

9. The oral disintegrating film of claim 8, wherein the excipient comprises citric acid.

10. The oral disintegrating film of claim 1, wherein the cannabinoid or cannabinoid analogue is present in the film in an amount of from about 0.5 mg to 100 mg.

11. The oral disintegrating film of claim 1, wherein the solubilizing agent is present in the formulation in an amount of from about 5% to 80% based on weight.

12. The oral disintegrating film of claim 7, wherein the excipient comprises a plasticizer selected from the group consisting of glycerin, medium chain glycerides (MCGs), long chain glycerides, propylene glycol esters and mixtures thereof.

13. The oral disintegrating film of claim 12, wherein the plasticizer comprises glycerin.

14. The oral disintegrating film of claim 1, wherein the oral disintegrating film has a thickness of from about 10 mm to 50 mm.

15. The oral disintegrating film of claim 1, wherein the oral disintegrating film has a weight of from about 30 mg to 200 mg.

16. The oral disintegrating film of claim 1, wherein the oral disintegrating film dissolves in less than 60 seconds in water at 23° C.

17. An oral disintegrating film comprising:
a cannabinoid or cannabinoid analogue;
a first film forming polymer;
a second film forming polymer; and
a solubilizing agent, wherein the oral disintegrating film self-emulsifies in an aqueous medium.

18. The oral disintegrating film of claim 17, wherein each of the first film forming polymer and the second film forming polymer is selected from the group consisting of gelatin, pectin, hydroxypropylmethyl cellulose, methoxypolyethylene glycols, polyethylene glycols, hydroxyethyl cellulose, hydroxypropyl cellulose, carboxymethyl cellulose, polyvinylalcohol, polyacrylic acid, methyl methacrylate copolymer, polyvinyl caprolactam-polyvinyl acetate-polyethylene glycol graft co-polymer, carboxyvinyl polymer, polyethyleneglycol, alginic acid, sodium alginate, modified starch, casein, whey protein extract, soy protein extract, pea protein, rice, millet, buckwheat, tapioca, carboxymethyl/hydroxypropyl dual-modified tapioca, gelatinized tapioca starch, gelatinized potato starch, potato starch hydrolysates, legumes, zein, levan, elsinan, gluten, acacia gum, carageenan, Arabic gum, guar gum, locust bean gum, xanthan gum, gellan gum, agar and mixtures thereof.

19. The oral disintegrating film of claim 18, wherein the first film forming polymer is selected from the group consisting of hydroxypropylmethyl cellulose, methoxypolyethylene glycols, Carbowax Sentry Polyethylene glycols, hydroxyethyl cellulose, hydroxypropyl cellulose, carboxymethyl cellulose, polyethylene glycols, hydroxyethyl cellulose, hydroxypropyl cellulose, carboxymethyl cellulose and mixtures thereof and the second film forming polymer is selected from the group consisting of gelatin, pectin, acacia gum, carageenan, Arabic gum, guar gum, locust bean gum, xanthan gum, gellan gum, agar and mixtures thereof.

20. A method of reducing side effects associated with chemotherapy or radiation treatment, alleviating pain or suppressing appetite in a subject in need thereof comprising administering to the subject the oral disintegrating film of claim 1.
